(19) United States
(12) Patent Application Publication
(10) Pub. No.: US 2015/0094223 A1
(43) Pub. Date: Apr. 2, 2015

(54) METHODS AND APPARATUSES FOR DIAGNOSING CANCER BY USING GENETIC INFORMATION

(71) Applicant: Samsung Electronics Co., Ltd., Suwon-si (KR)

(72) Inventors: Eunjin Lee, Seongnam-si (KR); Taejin Ahn, Seoul (KR)

(21) Appl. No.: 14/476,479

(22) Filed: Sep. 3, 2014

(30) Foreign Application Priority Data
Oct. 2, 2013 (KR) 10-2013-0118120

(51) Int. Cl. G06F 19/00; G06F 19/20; C12Q 1/68

(52) U.S. Cl. CPC G06F 19/34; C12Q 1/6886; G06F 19/20; C12Q 2600/158
USPC 506/9; 506/39; 702/20

(57) ABSTRACT

A method and apparatus for diagnosing cancer by using genetic information, the method comprising acquiring first gene expression data of a subject, for whom cancer is to be diagnosed, for a gene marker set including at least one gene marker; and determining a possibility of a presence of the cancer of the subject by using the acquired first gene expression data and pre-stored second gene expression data of a normal person group and a cancer patient group, wherein the gene marker set includes gene markers such as pyrroline-5-carboxylate reductase 1 (PYCR1), phosphoglycerate dehydrogenase (PHGDH), glutaminase 2 (liver, mitochondrial) (GLS2), and glutaminase (GLS) among others.

### CROSS-REFERENCE TO RELATED APPLICATIONS

This application claims the benefit of Korean Patent Application No. 10-2013-0118120, filed on Oct. 2, 2013, in the Korean Intellectual Property Office, the entire disclosure of which is hereby incorporated by reference.

### BACKGROUND OF THE INVENTION

1. Field

The present disclosure relates to methods and apparatuses for diagnosing diseases, such as cancer, by using genetic information of a subject.

2. Description of the Related Art

A genome denotes all genetic information of one organism. Various technologies, such as a deoxyribonucleic acid (DNA) chip and next generation sequencing technology are being developed to sequence a genome of a person. Genetic information, such as a nucleic acid sequence, is widely used to find genes associated with diseases, such as diabetes, cancer, etc., or to determine a correlation between genetic diversity and an expression characteristic of an individual. In particular, genetic information collected from a person is important in determining genetic features of a person associated with different symptoms or a progression of a disease. Therefore, genetic information, such as a nucleic sequence, of a person is fundamental data for determining current and future disease-related information to prevent a disease or selecting an optimal treatment method at the initial stage of a disease. Regarding genetic information of an organism, research is being done into technologies that accurately analyze genetic information of a person and diagnose a disease of the person by using a genome detection apparatus, which detects single nucleotide polymorphism (SNP) and copy number variation (CNV), such as a DNA chip, a microarray, or the like.

### BRIEF SUMMARY OF THE INVENTION

Provided are methods and apparatuses for diagnosing diseases, such as cancer, by using genetic information of a subject.

Provided is a non-transitory computer-readable storage medium storing a program for executing the methods.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram illustrating a cancer diagnostic system;

FIG. 2 is a table listing gene markers included in a gene marker set which is used in a cancer diagnostic apparatus;

FIG. 3 is a diagram illustrating gene markers belonging to a pathway of amino acid synthesis and interconversion (transamination) among gene networks associated with glutamate metabolism;

FIG. 4 is a diagram for describing a simulation method and result for discovering a gene pathway used to analyze a correlation between glutamate metabolism and the presence of a cancer (particularly, adenocarcinoma of the lung);

FIG. 5 is a diagram for describing a gene marker capable of contributing to an increase in the accuracy of cancer diagnosis;

FIG. 6 is a diagram for describing the number of gene markers capable of contributing to an increase in the accuracy of cancer diagnosis;

FIG. 7 is a block diagram of the cancer diagnostic apparatus;

FIG. 8 is a block diagram of a detailed configuration of a determination unit;

FIG. 9 is a graph showing a preprocessing result of first gene expression data which is preprocessed by a preprocessor;

FIG. 10 is a graph for describing an operation in which the determination unit determines a possibility of cancer of a subject by using a discriminant model;

FIG. 11 is a flowchart of a method of diagnosing cancer by using genetic information;

FIG. 12 is a flowchart of a method of diagnosing cancer by using genetic information; and

FIG. 13 is a flowchart of a method of diagnosing cancer by using genetic information.

### DETAILED DESCRIPTION OF THE INVENTION

Additional aspects will be set forth in part in the description which follows and, in part, will be apparent from the description, or may be learned by practice of the presented embodiments.

According to an aspect of the present invention, a method of diagnosing cancer by using genetic information includes: acquiring first gene expression data of an subject, for whom cancer is to be diagnosed, for a gene marker set including at least one gene marker; and determining a possibility of cancer of the subject by using the acquired first gene expression data and pre-stored second gene expression data of a normal person group and a cancer patient group, wherein the gene marker set includes at least one gene marker selected from the group consisting of pyrroline-5-carboxylate reductase 1 (PYCR1), phosphoglycerate dehydrogenase (PHGDH), glutaminase 2 (liver, mitochondrial) (GLS2), glutaminase (GLS), glutamate dehydrogenase 1 (GLUD1), glutamate-ammonia ligase (GLUL), glutamic-oxaloacetic transaminase 1 and soluble (aspartate aminotransferase 1) (GOT1), glutamic-oxaloacetic transaminase 2 and mitochondrial (aspartate aminotransferase 2) (GOT2), glutamic-pyruvate transaminase (alanine aminotransferase) (GPT), glutamic pyruvate transaminase (alanine aminotransferase 2) (GPT2), phosphoserine aminotransferase 1 (PSAT1), asparagine synthetase (glutamine-hydrolyzing) (ASNS), ornithine aminotransferase (OAT), phosphoserine phosphatase (PSPH), aldehyde dehydrogenase 18 family and member A1 (ALDH18A1), and cysteine conjugate-beta lyase cytoplasmic (CCBL1).

The gene marker set may include PYCR1, and comprise at least one gene marker selected from the group consisting of PHGDH, GLS2, GLS, GLUD1, GLUL, GOT1, GOT2, GPT, GPT2, PSAT1, ASNS, OAT, PSPH, ALDH18A1, and CCBL1.

The gene marker set may comprise one gene marker PYCR1.

The gene marker set may comprise all the gene markers PYCR1, PHGDH, GLS2, GLS, GLUD1, GLUL, GOT1, GOT2, GPT, GPT2, PSAT1, ASNS, OAT, PSPH, ALDH18A1, and CCBL1.

The method may further comprise preprocessing the first gene expression data including first gene expression levels, based on a distribution of second gene expression levels included in the pre-stored second gene expression data, wherein the determining may include determining the presence of cancer by using the preprocessed first gene expression data and the pre-stored second gene expression data.

The preprocessing may comprise calculating ratios of the second gene expression levels and the first gene expression levels in units of a gene marker to preprocess the first gene expression data.

The preprocessing may comprise normalizing or standardizing the first gene expression levels in units of a gene marker to preprocess the first gene expression data in comparison with the second gene expression levels.

The determining may comprise applying the preprocessed first gene expression data to a discriminant model, pre-generated from the pre-stored second gene expression data, to determine the possibility of cancer.

The discriminant model may be pre-generated by using a regression model, which has a univariate representing the gene marker set or a multi-variate corresponding to two or more of the gene markers included in the gene marker set, for the pre-stored second gene expression data.

The determining may include: calculating an index, indicating a degree of expression of the first gene expression levels in the preprocessed first gene expression data, for the second gene expression levels; and applying the calculated index to the pre-generated discriminant model to calculate a statistical significance level indicating a presence probability of cancer, and the possibility of cancer may be determined based on the calculated statistical significance level.

The calculating of an index may include calculating the index by using at least one of the following methods: a fisher exact test, a binomial test, a geneset enrichment analysis (GSEA), a Mahalanobis distance, a Euclid distance, a Manhattan distance, a maximum distance, a minimum distance, and a correlation coefficient.

The calculating of an index may include estimating a representative expression pattern that is obtained by summarizing distributions of third gene expression levels of the normal person group in the second gene expression data, and the index may be calculated based on the degree of expression of the first gene expression levels in the preprocessed first gene expression data for the estimated representative expression pattern.

The determining may include: calculating an index, indicating a degree of expression of the first gene expression levels in the preprocessed first gene expression data, for a representative expression pattern that is obtained by summarizing distributions of third gene expression levels of the normal person group; and calculating a statistical significance level indicated by the calculated index by using an empirical distribution of degrees of expression of the third gene expression levels for the representative expression pattern, and the possibility of cancer may be determined based on the calculated statistical significance level.

The determining may further include comparing the calculated statistical significance level and a threshold value, which is used to determine the presence of cancer or a degree of occurrence of cancer, by using the pre-generated discriminant model, and the possibility of cancer may be determined based on the compared result.

According to another aspect of the present disclosure, an apparatus for diagnosing cancer by using genetic information comprises: a gene expression data acquiring unit that acquires first gene expression data of an subject, for whom cancer is to be diagnosed, for a gene marker set including at least one gene marker; and a determination unit that determines a possibility of cancer of the subject by using the acquired first gene expression data and pre-stored second gene expression data of a normal person group and a cancer patient group, wherein the gene marker set includes at least one gene marker selected from the group consisting of pyrroline-5-carboxylate reductase 1 (PYCR1), phosphoglycerate dehydrogenase (PHGDH), glutaminase 2 (liver, mitochondrial) (GLS2), glutaminase (GLS), glutamate dehydrogenase 1 (GLUD1), glutamate-ammonia ligase (GLUL), glutamic-oxaloacetic transaminase 1 and soluble (aspartate aminotransferase 1) (GOT1), glutamic-oxaloacetic transaminase 2 and mitochondrial (aspartate aminotransferase 2) (GOT2), glutamic-pyruvate transaminase (alanine aminotransferase) (GPT), glutamic pyruvate transaminase (alanine aminotransferase 2) (GPT2), phosphoserine aminotransferase 1 (PSAT1), asparagine synthetase (glutamine-hydrolyzing) (ASNS), ornithine aminotransferase (OAT), phosphoserine phosphatase (PSPH), aldehyde dehydrogenase 18 family and member A1 (ALDH18A1), and cysteine conjugate-beta lyase cytoplasmic (CCBL1).

The gene marker set may comprise PYCR1, and include at least one gene marker selected from the group consisting of PHGDH, GLS2, GLS, GLUD1, GLUL, GOT1, GOT2, GPT, GPT2, PSAT1, ASNS, OAT, PSPH, ALDH18A1, and CCBL1.

The determination unit may comprise a preprocessor that preprocesses the first gene expression data including first gene expression levels, based on a distribution of second gene expression levels included in the pre-stored second gene expression data, and the determination unit may determine the presence of cancer by using the preprocessed first gene expression data and the pre-stored second gene expression data.

The apparatus may further comprise a storage unit that stores a discriminant model pre-generated from the pre-stored second gene expression data, wherein the determination unit may determine the possibility of cancer by using the pre-generated discriminant model and the preprocessed first gene expression data.

The determination unit may further include a calculator that calculates an index, indicating a degree of expression of the first gene expression levels in the preprocessed first gene expression data, for the second gene expression levels, and applies the calculated index to the pre-generated discriminant model to calculate a statistical significance level indicating a presence probability of cancer, and the determination unit may determine the possibility of cancer, based on the calculated statistical significance level.

The determination unit may further include a comparator that compares the calculated statistical significance level and a threshold value, which is used to determine the presence of cancer or a degree of occurrence of cancer, by using the pre-generated discriminant model, and the determination unit may determine the possibility of cancer, based on the compared result.

According to another aspect of the present disclosure, a method of detecting cancer using genetic information, the method comprising: acquiring first gene expression data of a subject for a gene marker set including at least one gene marker; and comparing the acquired first gene expression data to pre-stored second gene expression data of a normal person group and a cancer patient group by calculating statistical similarity of the first gene expression data to the pre-stored second gene expression data, wherein cancer in the subject is indicated if the first gene expression data is more similar to the pre-stored second gene expression data of the cancer patient group than to the pre-stored second gene expression data of the normal patient group, and wherein the gene marker set comprises at least one gene marker selected from the group consisting of pyrroline-5-carboxylate reductase 1 (PYCR1), phosphoglycerate dehydrogenase (PHGDH), glutaminase 2 (liver, mitochondrial) (GLS2), glutaminase (GLS), glutamate dehydrogenase 1 (GLUD1), glutamate-ammonia ligase (GLUL), glutamic-oxaloacetic transaminase 1 and soluble (aspartate aminotransferase 1) (GOT1), glutamic-oxaloacetic transaminase 2 and mitochondrial (aspartate aminotransferase 2) (GOT2), glutamic-pyruvate transaminase (alanine aminotransferase) (GPT), glutamic pyruvate transaminase (alanine aminotransferase 2) (GPT2), phosphoserine aminotransferase 1 (PSAT1), asparagine synthetase (glutamine-hydrolyzing) (ASNS), ornithine aminotransferase (OAT), phosphoserine phosphatase (PSPH), aldehyde dehydrogenase 18 family and member A1 (ALDH18A1), and cysteine conjugate-beta lyase cytoplasmic (CCBL1), wherein the statistical similarity is calculated using at least one of a fisher exact test, a binomial test, a geneset enrichment analysis (GSEA), a Mahalanobis distance, a Euclid distance, a Manhattan distance, a maximum distance, a minimum distance, and a correlation coefficient.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

Hereinafter, embodiments of the present invention will be described in detail with reference to the accompanying drawings.

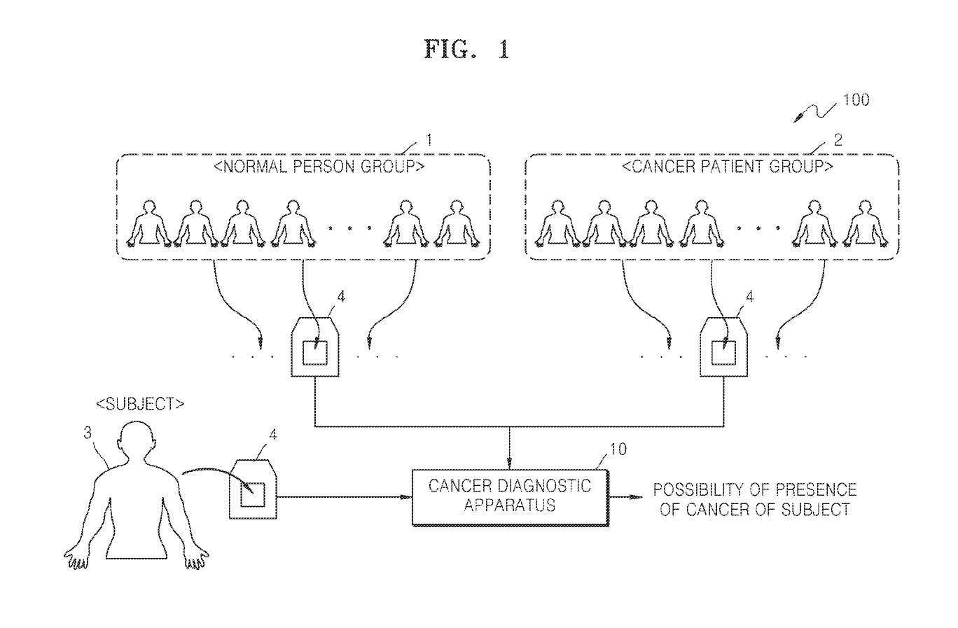

FIG. 1 is a diagram illustrating a cancer diagnostic system 100 according to an embodiment of the present invention. Referring to FIG. 1, the cancer diagnostic system 100 includes a cancer diagnostic apparatus 10, a normal person group 1, a cancer patient group 2, and a plurality of microarrays 4 for analyzing genetic information of a subject 3. Here, the cancer patient group 2 is a group of persons diagnosed with cancer, and the subject 3 is a person who is to be diagnosed for a possibility of cancer by using the cancer diagnostic apparatus 10.

Although not shown in FIG. 1, it can be understood by one of ordinary skill in the art that the cancer diagnostic system 100 may further include an image analysis apparatus, such as a high content cell imaging apparatus, a high content screening apparatus, or a high throughput screening apparatus, for detecting a gene expression pattern or a gene expression level from the normal person group 1, the cancer patient group 2, or the subject 3, and a polymerase chain reaction (PCR) apparatus may be used instead of the microarrays 4.

That is, the cancer diagnostic system 100 of FIG. 1 is illustrated as including only elements relating to the present embodiment so as to prevent the feature of the present embodiment from being obscured, but may further include other general-use elements than the elements of FIG. 1.

A nucleic acid, such as a DNA, of an individual corresponds to a genetic material (i.e., a gene) including genetic information. A nucleic acid sequence includes information about tissue and cells constituting an individual. Therefore, research into information about a complete nucleic acid sequence of a person is frequently carried out while investigating a vital phenomenon, developing a new drug, diagnosing and preventing a disease, and researching the heredity of humans.

Recently, due to the advance of genome research, a functional correlation between genes included in a genome has gradually been disclosed, and thus, an analysis of a gene network between genes is attracting much attention. This may be because most physiological phenomena happening in an organism are caused by a reaction between a plurality of genes instead of one gene.

The gene network is expressed as a network in which genes are intricately connected to each other, and may be acquired from a database, known by one of ordinary skill in the art, such as the national center for biotechnology information (NCBI). However, due to the advance of gene analysis technology, a new gene network is continuously disclosed, and the existing gene network is updated with the new gene network. Therefore, a gene network to be described in the present embodiment is not limited to the gene network acquired from the known database.

In the cancer diagnostic system 100, as described above, the cancer diagnostic apparatus 10 performs an analysis based on a correlation between genetic information and the cancer occurrence of a person to increase an efficiency and accuracy of cancer diagnosis.

To date, a correlation between various kinds of cancers and a gene pathway was elucidated through much research into a genome of a person. Among the expansive research, there is research indicating glutamate metabolism is relevant to cancer metabolism. Such research is disclosed in the paper of Munoz-Pinedo C. et al. published in the Cell Death and Disease Journal (2012) and the paper of Kara L. Cerveny published in the Cell Journal (2013). Thus, an influence of glutamate metabolism on cancer metabolism is not described here in detail.

Glutamate metabolism is metabolism that greatly affects the growth or metastasis of a cancer cell, and particularly affects a process in which glutamine is changed to alpha keto glutamate corresponding to an energy source and a building block of a cancer cell.

According to the present embodiment, the cancer diagnostic apparatus 10 uses, as gene markers (e.g., bio markers), various kinds of gene networks associated with glutamate metabolism and genes belonging to a gene pathway highly associated with the occurrence of cancer among a plurality of gene pathways in diagnosing cancer, thus increasing the accuracy or efficiency of cancer diagnosis.

In particular, the cancer diagnostic apparatus 10 may use, as a gene marker set, genes belonging to a pathway of amino acid synthesis and interconversion (transamination) among various kinds of gene networks associated with glutamate metabolism.

In more detail, when the genes belonging to the pathway of amino acid synthesis and interconversion (transamination) are abnormal, the cancer diagnostic apparatus 10 may determine glutamate metabolism as being abnormal. Furthermore, when glutamate metabolism is abnormal, the cancer diagnostic apparatus 10 may determine there to be a high possibility of lung cancer (adenocarcinoma of the lung).

However, it can be understood by one of ordinary skill in the art that the present embodiment is not limited to lung cancer (adenocarcinoma of the lung), and may be used to diagnose various kinds of cancers including but not limited to breast cancer, colon cancer, ovarian cancer, etc.

That is, the cancer diagnostic apparatus 10 may determine whether the genes belonging to the pathway of amino acid synthesis and interconversion (transamination) are abnormal, and determine the presence of cancer or a degree of cancer occurrence.

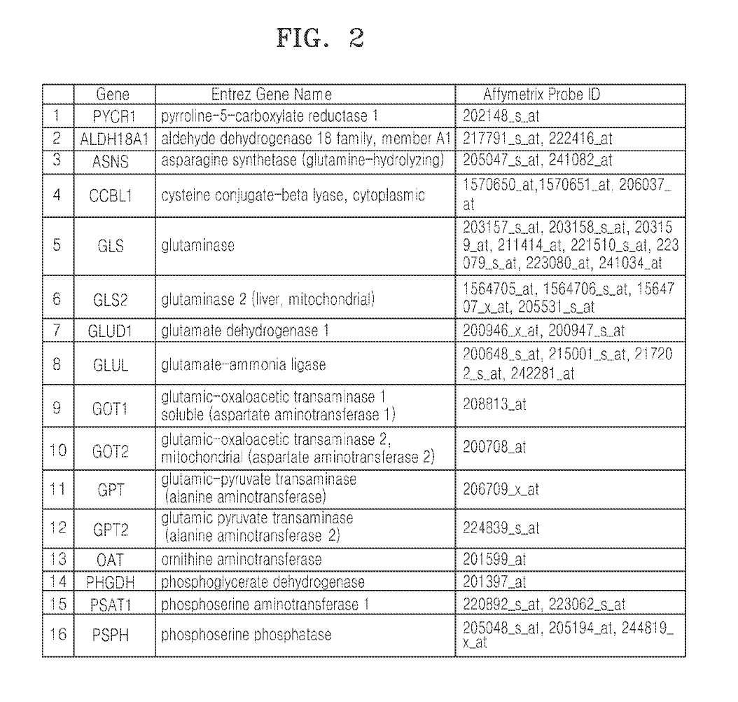

FIG. 2 is a table listing gene markers included in a gene marker set which is used in the cancer diagnostic apparatus 10, according to an embodiment of the present disclosure.

Referring to FIG. 2, the gene marker set used in the present embodiment may include at least one gene marker selected from the group consisting of pyrroline-5-carboxylate reductase 1 (PYCR1), phosphoglycerate dehydrogenase (PHGDH), glutaminase 2 (liver, mitochondrial) (GLS2), glutaminase (GLS), glutamate dehydrogenase 1 (GLUD1), glutamate-ammonia ligase (GLUL), glutamic-oxaloacetic transaminase 1 and soluble (aspartate aminotransferase 1) (GOT1), glutamic-oxaloacetic transaminase 2 and mitochondrial (aspartate aminotransferase 2) (GOT2), glutamic-pyruvate transaminase (alanine aminotransferase) (GPT), glutamic pyruvate transaminase (alanine aminotransferase 2) (GPT2), phosphoserine aminotransferase 1 (PSAT1), asparagine synthetase (glutamine-hydrolyzing) (ASNS), ornithine aminotransferase (OAT), phosphoserine phosphatase (PSPH), aldehyde dehydrogenase 18 family and member A1 (ALDH18A1), and cysteine conjugate-beta lyase cytoplasmic (CCBL1). That is, the gene marker set used in the present embodiment may include all of the above-described sixteen gene markers, or include one or more gene markers. As described above, the sixteen gene markers correspond to the genes belonging to the pathway of amino acid synthesis and interconversion (transamination).

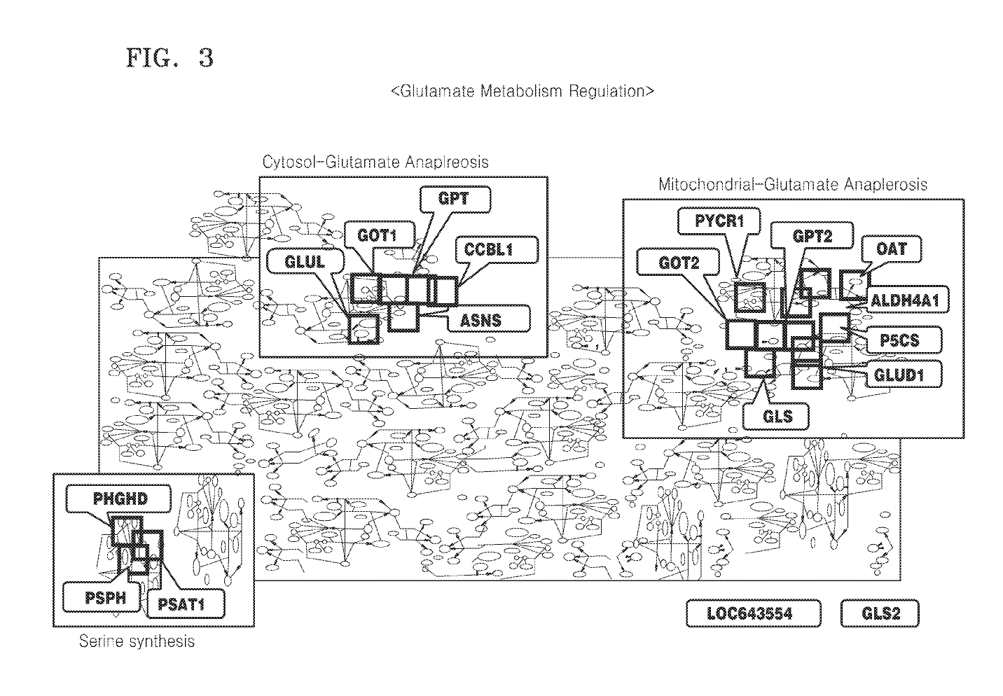

FIG. 3 is a diagram illustrating gene markers belonging to the pathway of amino acid synthesis and interconversion (transamination) among gene networks associated with glutamate metabolism, according to an embodiment of the present invention.

Referring to FIG. 3, the gene markers PYCR1, PHGDH, GLS2, GLS, GLUD1, GLUL, GOT1, GOT2, GPT, GPT2, PSAT1, ASNS, OAT, PSPH, ALDH18A1, and CCBL1, associated with regulation of glutamate metabolism and described above with reference to FIG. 2, are relevant to mechanisms such as cytosol-glutamate anaplerosis, mitochondrial-glutamate anaplerosis, serine synthesis, etc.

Regarding glutamate metabolism, as described above, various kinds of gene networks or gene pathways intervene in the occurrence of cancer (e.g., adenocarcinoma of the lung).

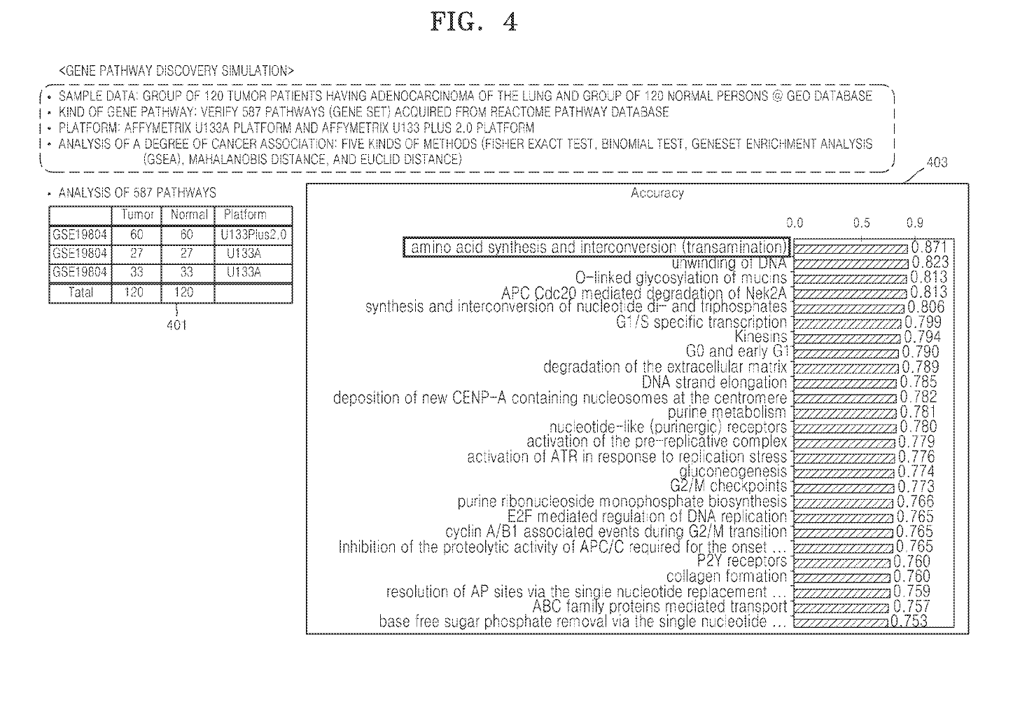

According to the present embodiment, however, the pathway of amino acid synthesis and interconversion (transamination) among the gene pathways is used. The reason will be described with reference to a simulation result of FIG. 4.

FIG. 4 is a diagram for describing a simulation method and result for discovering a gene pathway used to analyze a correlation between glutamate metabolism and the presence of cancer (e.g., adenocarcinoma of the lung), according to an embodiment of the present invention.

Referring to FIG. 4, the simulation method for discovering a gene pathway uses gene expression data of a group of 120 tumor patients having adenocarcinoma of the lung and gene expression data of a group of 120 normal persons, which are acquired from a gene expression omnibus (GEO) database.

Here, 587 gene pathways acquired from reactome that is a database of biological pathways are selected as gene pathways to verify for gene expression data of a cancer patient group and a normal person group.

Regarding the 587 gene pathways, gene expression data is analyzed by using an Affymetrix U133A platform and an Affymetrix U133 plus 2.0 platform.

In a detailed simulation setting, as shown in a table 401, among gene expression data of a group of 60 lung cancer patients and gene expression data of a group of 60 normal persons which are acquired a GSE19804 data set (DataSet) of the GEO database, the selected 587 gene pathways are analyzed by using Affymetrix U133 plus 2.0 platform. Furthermore, among gene expression data of a group of 27 lung cancer patients and gene expression data of a group of 27 normal persons which are acquired the GSE19804 data set (DataSet), the selected 587 gene pathways are analyzed by using the Affymetrix U133A platform. Also, among gene expression data of a group of 33 lung cancer patients and gene expression data of a group of 33 normal persons which are acquired the GSE19804 data set (DataSet), the selected 587 gene pathways are analyzed by using the Affymetrix U133A platform.

In this case, in order to analyze a degree of association between cancer and the 587 gene pathways, a degree of association between a gene pathway and a possibility of lung cancer is analyzed or simulated by using five kinds of methods, namely, fisher exact test, binomial test, geneset enrichment analysis (GSEA), Mahalanobis distance, and Euclid distance.

The simulation result shows which among various gene pathways such as the pathway of amino acid synthesis and interconversion (transamination), a pathway of unwinding of DNA, a pathway of O-linked glycosylation of mucins, a pathway of APC Cdc20 mediated degradation of Nek2A, a pathway of synthesis and interconversion of nucleotide di- and triphosphates, a pathway of G1/S specific transcription, and a pathway of kinesins, the pathway of amino acid synthesis and interconversion (transamination) has the highest accuracy of 0.871.

In other words, the simulation result denotes that among various gene pathways associated with glutamate metabolism, a gene pathway having the highest correlation with a possibility of cancer is the pathway of amino acid synthesis and interconversion (transamination).

Therefore, according to the present embodiment, since at least one gene marker selected from the group consisting of PYCR1, PHGDH, GLS2, GLS, GLUD1, GLUL, GOT1, GOT2, GPT, GPT2, PSAT1, ASNS, OAT, PSPH, ALDH18A1, and CCBL1 which are belonging to the pathway of amino acid synthesis and interconversion (transamination) is used as described above with reference to FIG. 2, a possibility of cancer (particularly, adenocarcinoma of the lung) is diagnosed more accurately and efficiently than a case using other gene pathways associated with glutamate metabolism.

Moreover, an accuracy of cancer diagnosis is increased by a specific gene marker. Alternatively, an accuracy of cancer diagnosis is increased according to the number of gene markers included in the gene marker set.

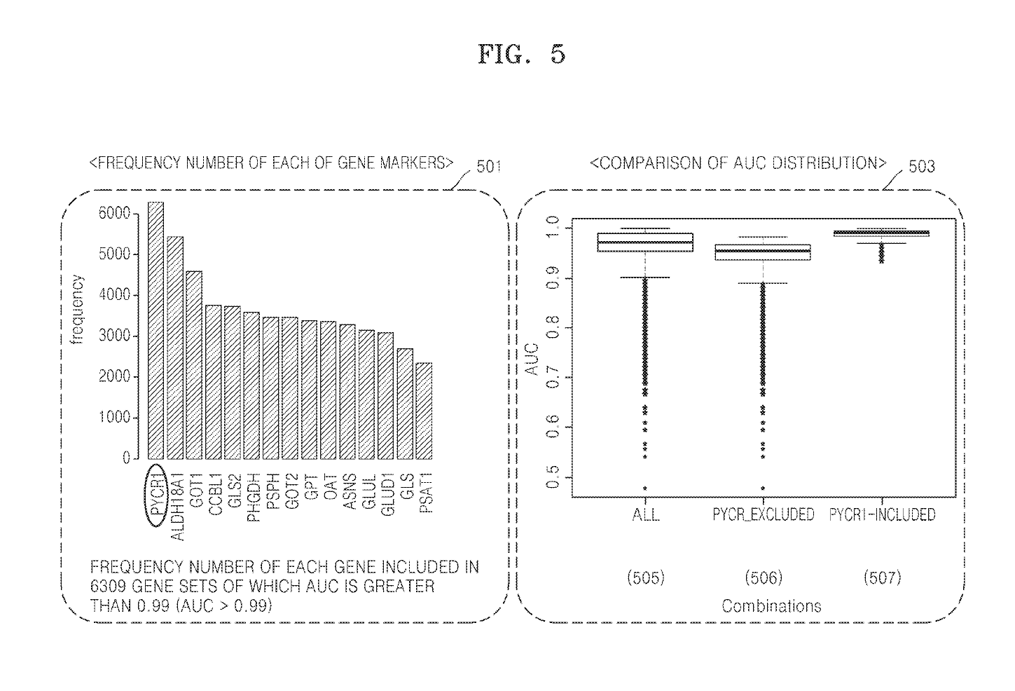

FIG. 5 is a diagram for describing a gene marker capable of contributing to an increase an accuracy of cancer diagnosis according to an embodiment of the present disclosure.

Referring to FIG. 5, a graph 501 showing the frequency number of genes included in 6309 gene marker sets analyzed as an area under the curve (AUC) being 0.99 or more is shown as an analysis result of a correlation with the presence of cancer for the number of cases of all gene marker sets which are obtained by combining the sixteen gene markers PYCR1, PHGDH, GLS2, GLS, GLUD1, GLUL, GOT1, GOT2, GPT, GPT2, PSAT1, ASNS, OAT, PSPH, ALDH18A1, and CCBL1.

As a result, the frequency number of the gene marker PYCR1 is shown as being highest, which denotes that when the gene marker PYCR1 is included in a gene marker set for cancer diagnosis, an accuracy of cancer diagnosis increases.

Referring to an AUC distribution graph 503, a result, which is obtained by comparing an AUC distribution of a gene marker set 505 including all the sixteen gene markers PYCR1, PHGDH, GLS2, GLS, GLUD1, GLUL, GOT1, GOT2, GPT, GPT2, PSAT1, ASNS, OAT, PSPH, ALDH18A1, and CCBL1, an AUC distribution of gene marker sets 506 which are obtained by combining fifteen gene markers except PYCR1, and an AUC distribution of gene marker sets 507 which are obtained by combining the sixteen gene markers including PYCR1, shows that the AUC distribution of the gene marker sets 506 is lower than the AUC distribution of the gene marker set 505 and the AUC distribution of the gene marker sets 507.

That is, similarly to the analysis of the graph 501, an analysis of the AUC distribution graph 503 may denote that when the gene marker PYCR1 is included in the gene marker set for cancer diagnosis, an accuracy of cancer diagnosis increases.

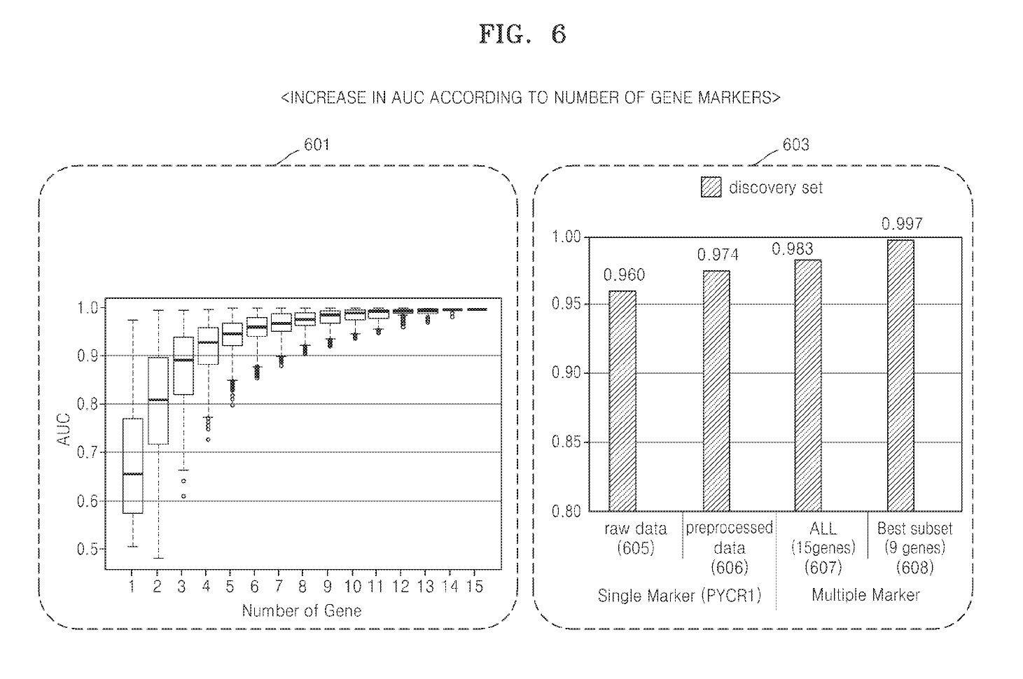

FIG. 6 is a diagram for describing the number of gene markers capable of contributing to an increase in the accuracy of cancer diagnosis, according to an embodiment of the present disclosure.

Referring to an AUC distribution graph 601 shown at a left side of FIG. 6, an AUC progressively increases from a case (in which the number of gene markers is one) to a case in which the number of gene markers is fifteen. That is, it can be seen that as the number of gene markers included in a gene marker set for cancer diagnosis increases, an accuracy of cancer diagnosis increases.

Referring to an AUC distribution graph 603 shown at a right side of FIG. 6, it can be seen that an AUC value increases in the order of an AUC of a gene marker set 605 of raw data, an AUC of a gene marker set 606 of preprocessed data, an AUC of a gene marker set 607 including all the sixteen gene markers, and an AUC of a gene marker set 608 including gene markers of nine selected best subsets. That is, it can be seen that an accuracy of cancer diagnosis increases more when using a gene marker set (607 or 608) including a number of gene markers than when using a gene marker set (605 or 606) including only one gene marker PYCR1.

Here, a discovery set of the AUC distribution graph 603 denotes a plurality of pieces of the gene expression data used in the simulation of FIG. 4. The meaning of the preprocessed data will be described below in a corresponding part.

To summarize the descriptions above with reference to FIGS. 1 to 6, the cancer diagnostic system 100 according to the present embodiment is a system that diagnoses cancer by using an association between glutamate metabolism and cancer metabolism.

In particularly, as described above with reference to FIG. 4, the cancer diagnostic system 100 according to the present embodiment is a system that uses the pathway of amino acid synthesis and interconversion (transamination) having the highest association with cancer metabolism among various kinds of gene pathways associated with glutamate metabolism. In more detail, as described above with reference to FIG. 2, the cancer diagnostic system 100 is a system that diagnoses cancer by using a gene marker set including at least one gene marker selected from the group consisting of PYCR1, PHGDH, GLS2, GLS, GLUD1, GLUL, GOT1, GOT2, GPT, GPT2, PSAT1, ASNS, OAT, PSPH, ALDH18A1, and CCBL1 which are in the pathway of amino acid synthesis and interconversion (transamination).

Moreover, as described above, when gene markers are combined in order for a gene marker set to at least include PYCR1, the accuracy of cancer diagnosis increases.

In addition, as described above with reference to FIG. 6, an accuracy of cancer diagnosis increases more when a gene marker set includes a number of gene markers than when the gene marker set includes a small number of gene markers.

Hereinafter, a function and an operation of the cancer diagnostic apparatus 10 for diagnosing cancer of the subject 3 by using the gene markers will be described in more detail.

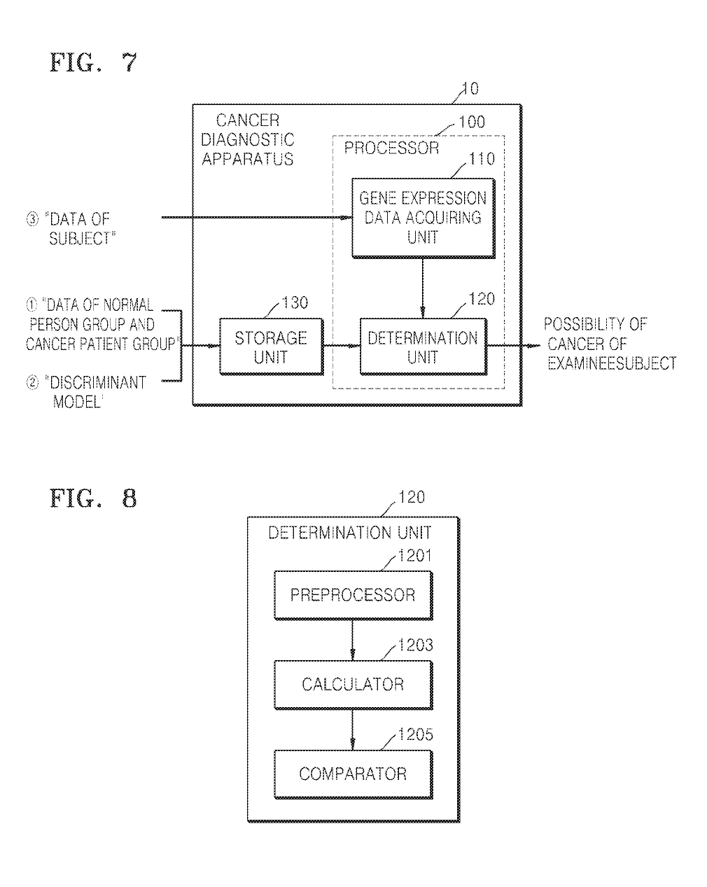

FIG. 7 is a block diagram of the cancer diagnostic apparatus 10 according to an embodiment of the present invention.

Referring to FIG. 7, the cancer diagnostic apparatus 10 includes a gene expression data acquiring unit 110, a determination unit 120, and a storage unit 130.

In the cancer diagnostic apparatus 10, the gene expression data acquiring unit 110 and the determination unit 120 may be implemented as a generally-used processor 100. That is, the processor 100 may be implemented with an array of a plurality of logic gates, or may be implemented as a combination of a general-use microprocessor and a memory which stores a program executed in the microprocessor. Also, the processor 100 may be implemented as a module type of an application program. Furthermore, it can be understood by one of ordinary skill in the art that the processor 100 may be implemented as hardware capable of performing operations to be described in the present embodiment.

The cancer diagnostic apparatus 10 of FIG. 7 is illustrated as including only elements relating to the present embodiment so as to prevent the feature of the present embodiment from being obscured, but may further include other general-use elements than the elements of FIG. 7.

The gene expression data acquiring unit 110 acquires first gene expression data of the subject 3 for a gene marker set including at least one gene marker.

Here, as described above, the gene marker set may include at least one gene marker selected from the group consisting of PYCR1, PHGDH, GLS2, GLS, GLUD1, GLUL, GOT1, GOT2, GPT, GPT2, PSAT1, ASNS, OAT, PSPH, ALDH18A1, and CCBL1. Alternatively, the gene marker set necessarily includes the gene marker PYCR1, and may further include at least one gene marker selected from the group consisting of PHGDH, GLS2, GLS, GLUD1, GLUL, GOT1, GOT2, GPT, GPT2, PSAT1, ASNS, OAT, PSPH, ALDH18A1, and CCBL1. Alternatively, the gene marker set may include only one gene marker PYCR1. Alternatively, the gene marker set may include all the gene markers PYCR1, PHGDH, GLS2, GLS, GLUD1, GLUL, GOT1, GOT2, GPT, GPT2, PSAT1, ASNS, OAT, PSPH, ALDH18A1, and CCBL1. That is, it can be seen by one of ordinary skill in the art that a combination of gene markers included in the gene marker set according to the present embodiment is not limited thereto.

The first gene expression data acquired by the gene expression data acquiring unit 110 may be image data that is obtained by analyzing biological samples, which are collected from the subject 3 and have undergone a hybridization reaction in the microarray 4, in the image analysis apparatus such as a high content cell imaging apparatus, a high content screening apparatus, or a high throughput screening apparatus. Alternatively, the first gene expression data may be statistical data that is obtained by digitizing a plurality of pieces of gene expression data analyzed from the image data.

A detailed operation, which acquires expression data from the biological samples by using the microarray 4 and the image analysis apparatuses, is known to one of ordinary skill in the art, and thus, its detailed description is not provided.

The storage unit 130 pre-stores second gene expression data of the normal person group 1 and the cancer patient group 2 and a discriminant model that is pre-generated from the second gene expression data.

The discriminant model is a model used to determine a possibility of cancer. When the second gene expression data is divided into gene expression data of the normal person group 1 and gene expression data of the cancer patient group 2, the discriminant model is a statistical model that analyzes and predicts into which of the normal person group 1 and the cancer patient group 2 individual gene expression levels included in the second gene expression data are classified.

Therefore, by using the discriminant model, when arbitrary gene expression data (for example, the first gene expression data of the subject 3) is newly inputted (acquired), whether the input gene expression data belongs to the normal person group 1 or the cancer patient group 2 may be analyzed by using a statistical probability.

The discriminant model may be generated by using a regression model, which has a univariate representing a gene marker set or a multi-variate corresponding to two or more gene markers, and the second gene expression data. Here, the regression model may be a model based on logistic regression. Such a logistic regression model having the univariate is expressed as the following Equation (1):

log it(p)=a₁·X_{Gene Marker Set}+a₂ (1)

where a1 denotes a coefficient, a2 denotes a coefficient, and XGene Marker Set denotes an independent variable. Also, log it(p) is a dependent variable, and denotes a probability that gene expression data is classified into the normal person group 1 (or the cancer patient group 2).

A logistic regression model having the multi-variate is expressed as the following Equation (2):

log it(p)=b₁·X_{PYCR1}+b₂·X_{ALDH18A1}+ . . . +bₙ·X_{Gene Marker} (2)

where each of b₁, b₂, . . . , bₙ denotes a coefficient, and each of XPYCR1 and XALDH18A1 denotes an independent variable corresponding to a gene marker. Also, the number of independent variables may be equal to or less than the number of gene markers included in a gene marker set, and log it(p), a dependent variable, denotes a probability that gene expression data is classified into the normal person group 1 (or the cancer patient group 2).

An index value to be calculated by a below-described calculator 1203 may be substituted into the independent variables of Equations (1) and (2).

An operation, which generates a regression model for each of the two groups (the normal person group 1 and the cancer patient group 2) with data given thereto as in Equations (1) and (2), is known to one of ordinary skill in the art, and thus, its detailed description is not provided.

In more detail, the discriminant model may be generated by using the logistic regression model, but is not limited thereto. It can be understood by one of ordinary skill in the art that the discriminant model may be generated by using another statistical analysis method such as analysis of variance (ANOVA) or correlation analysis.

That is, as described above, the storage unit 130 pre-stores the pre-generated discriminant model and the second gene expression data of the normal person group 1 and the cancer patient group 2 based on generation of the discriminant model.

The determination unit 120 determines a possibility of cancer of the subject 3 by using the second gene expression data of the normal person group 1 and the cancer patient group 2 and the first gene expression data of the subject 3. Here, the determination unit 120 may determine the presence of cancer as the determined result. Also, the determination unit 120 may determine a degree of occurrence of cancer, for example, a high risk or a low risk, as the determined result.

A detailed operation of determining a possibility of cancer in the determination unit 120 will be described in detail with reference to FIG. 8.

FIG. 8 is a block diagram of a detailed configuration of the determination unit 120 according to an embodiment of the present invention.

Referring to FIG. 8, the determination unit 120 includes a preprocessor 1201, a calculator 1203, and a comparator 1205.

The determination unit 120 of FIG. 8 is illustrated as including only elements relating to the present embodiment so as to prevent the feature of the present embodiment from being obscured, but may further include other general-use elements than the elements of FIG. 8.

First, the determination unit 120 reads out the second gene expression data, acquired from the normal person group 1 and the cancer patient group 2, and the discriminant model from the storage unit 130.

The preprocessor 1201 preprocesses the first gene expression data including first gene expression levels on the basis of a distribution of the second gene expression levels included in the second gene expression data.

To provide a more detailed description, the preprocessor 1201 may calculate ratios of the second gene expression levels and the first gene expression levels in units of a gene marker, thereby preprocessing the first gene expression data. Such a preprocessing method may be applied to a case in which the first gene expression data or the second gene expression data is data obtained by using a real-time polymerase chain reaction (RT-PCR), but is not limited thereto.

Moreover, the preprocessor 1201 may normalize or standardize the first gene expression levels in comparison with the second gene expression levels, thereby preprocessing the second gene expression data. Such a preprocessing method may be applied to a case in which the first gene expression data or the second gene expression data is data obtained by using the microarray 4, but is not limited thereto.

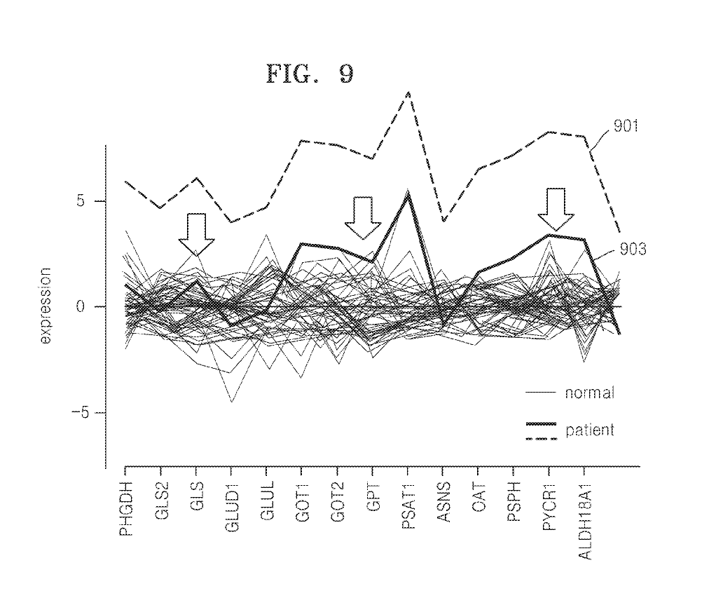

FIG. 9 is a graph showing a preprocessing result of the first gene expression data which is preprocessed by the preprocessor 1201, according to an embodiment of the present invention.

In FIG. 9, the second gene expression data of the normal person group 1 and the cancer patient group 2, pre-stored in the storage unit 130, may be preprocessed to have a pattern normalized or standardized with respect to a gene expression level of 0, or may be preprocessed by the preprocessor 1201, but is not limited thereto.

However, the newly acquired first gene expression data of the subject 3 is raw data 901, and is accurately analyzed only when preprocessing the first gene expression data in comparison with the second gene expression data. The preprocessor 1201 converts the raw data 901 of the first gene expression data into preprocessed first gene expression data 903 so as to have a pattern normalized or standardized with respect to the gene expression level of 0 as in the second gene expression data of the normal person group 1 and the cancer patient group 2.

The calculator 1203 calculates an index, indicating a degree of expression of the preprocessed first gene expression levels, for the second gene expression levels.

To describe an embodiment of an operation of calculating the index, the calculator 1203 may calculate the index by using the method such as the fisher exact test, the binomial test, the geneset enrichment analysis (GSEA), or the like.

In another embodiment of an operation of calculating the index, first, the calculator 1203 estimates a representative expression pattern that is obtained by summarizing distributions of gene expression levels of the normal person group 1 among the second gene expression data. The calculator 1203 calculates a representative value (or a centroid) based on an average, a weight average, a median value of gene expression levels for each gene marker, thereby estimating a representative expression pattern of each gene marker.

Subsequently, the calculator 1203 calculates an index for the estimated representative expression pattern on the basis of a degree of expression of the preprocessed first gene expression levels. Here, the calculator 1203 may calculate and analyze the index by using the Mahalanobis distance, the Euclid distance, the Manhattan distance, the maximum distance, the minimum distance, or a correlation coefficient. In addition, by using various methods, the calculator 1203 may calculate the index indicating a degree of expression.

The calculator 1203 applies or substitutes the calculated index into the discriminant model read out from the storage unit 130 to calculate a statistical significance level indicating a presence probability of cancer.

The comparator 1205 compares the statistical significance level calculated by the calculator 1203 and a threshold value, which is used to determine the presence of cancer or a degree of occurrence (for example, the high risk or the low risk) of cancer.

As a result, the determination unit 20 may determine a possibility of cancer depending on whether glutamate metabolism of the subject 3 is abnormal, on the basis of the comparison result from the comparator 1205.

The above-described index, statistical significance level, or threshold value may be one of a probability, a cumulative probability, a priority, a quantile, and a deviation.

Furthermore, the calculator 1203 may calculate the statistical significance level indicating a presence probability of cancer even without the discriminant model stored in the storage unit 130.

The calculator 1203, as described above, estimates the representative expression pattern that is obtained by summarizing the distributions of the gene expression levels of the normal person group 1.

Subsequently, the calculator 1203 calculates the index, indicating a degree of expression of the preprocessed first gene expression levels, for the estimated representative expression pattern. That is, an operation of calculating the index may be similar to the above-described operation.

Then, the calculator 1203 calculates the statistical significance level indicated by the calculated index by using an empirical distribution of degrees of expression of gene expression levels of the normal person group 1 for the estimated representative expression pattern, instead of the discriminant model. Here, the present embodiment is not limited to the empirical distribution, and may use another kind of null distribution.

The comparator 1205 compares the statistical significance level calculated by the calculator 1203 and the threshold value which is used to determine the presence of cancer or a degree of occurrence of cancer. The determination unit 20 may determine a possibility of cancer of the subject 3 on the basis of the comparison result from the comparator 1205.

According to the present embodiment, in order to calculate the statistical significance level, the discriminant model may be used, or instead of the discriminant model, the empirical distribution may be used. That is, the present embodiment is not limited thereto.

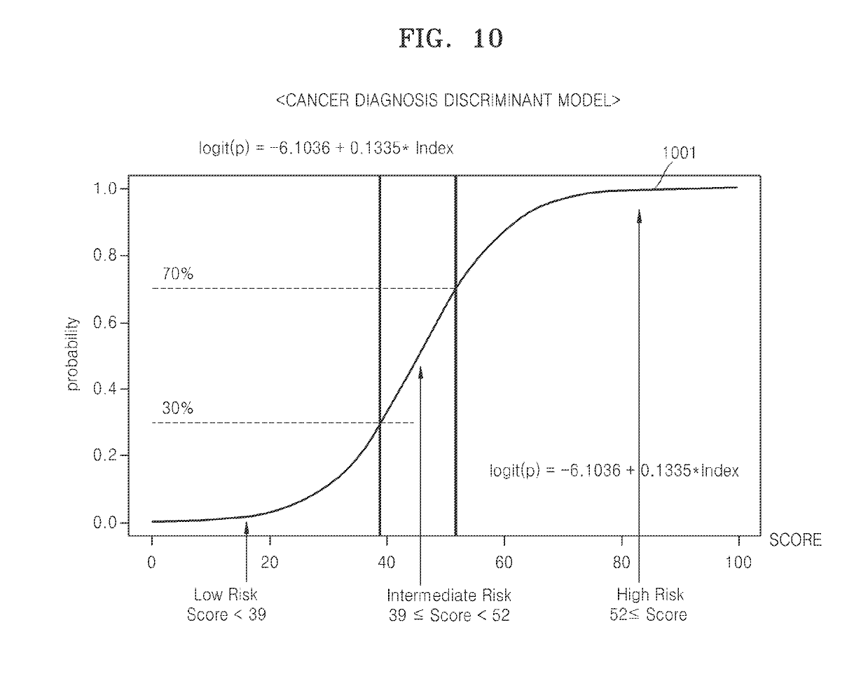

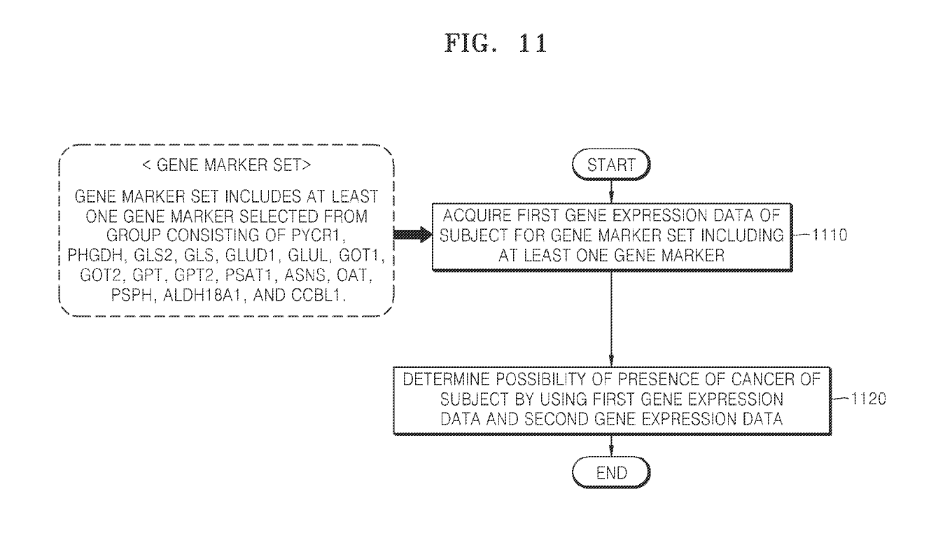

FIG. 10 is a graph for describing an operation in which the determination unit 120 determines a possibility of cancer of the subject 3 by using the discriminant model, according to an embodiment of the present disclosure.

Referring to FIG. 10, for example, it may be assumed that the discriminant model is pre-generated as expressed in Equation (3), based on an arbitrary simulation result of samples of the gene expression data of the group of 120 tumor patients having adenocarcinoma of the lung and the gene expression data of the group of 120 normal persons, which are acquired from the GEO database of FIG. 4.

Here, the pre-generated discriminant model may be pre-stored in the storage unit 130.

The following Equation (3) is merely an example which is arbitrarily assumed for convenience of description, and the present embodiment is not limited thereto. It can be understood by one of ordinary skill in the art that various discriminant models may be applied by using various kinds of samples.

log it(p)=−6.1036+0.1335·Index (3)

where Index denotes an index calculated by the calculator 1203. A regression curve 1001 may be generated according to the discriminant model expressed as Equation (3).

In FIG. 10, a value of log it(p) corresponds to a score, which denotes a statistical significance level indicating a presence probability of cancer.

Therefore, the determination unit 120 determines a possibility of cancer according to a section including the score, on the basis of a comparison result of the score and threshold values 39 and 52 compared by the comparator 1205. For example, when score<39, the determination unit 120 determines the subject 3 as having low-risk cancer, and when 39≦score<52, the determination unit 120 determines the subject 3 as having intermediate-risk cancer. Also, when 52≦score, the determination unit 120 determines the subject 3 as having high-risk cancer.

In FIG. 10, the threshold values have been described as two, but are not limited thereto. For example, one or more threshold values may be provided according to a cancer diagnosing condition. That is, it can be understood by one of ordinary skill in the art that a case in which the number of threshold values is one may be set when determining only the presence of cancer, and a case in which the number of threshold values is two or more may be set when further subdividing a process of determining a possibility of cancer to effect determination.

Moreover, it can be understood by one of ordinary skill in the art that a threshold value is not limited to the threshold values described above with reference to FIG. 10, and may be variously changed according to a cancer diagnosing condition.

FIG. 11 is a flowchart of a method of diagnosing cancer by using genetic information, according to an embodiment of the present invention. Referring to FIG. 11, the cancer diagnosis method according to the present embodiment includes a plurality of operations which are performed in a time series in the cancer diagnostic system 100 and the cancer diagnostic apparatus 10 illustrated in FIGS. 1, 7, and 8. Therefore, although not described below, descriptions above with reference to the drawings may be applied to the cancer diagnosis method according to the present embodiment.

In operation 1110, the gene expression data acquiring unit 110 acquires the first gene expression data of the subject 3 for a gene marker set including at least one gene marker.

The gene marker set used in operation 1110 includes at least one gene marker selected from the group consisting of PYCR1, PHGDH, GLS2, GLS, GLUD1, GLUL, GOT1, GOT2, GPT, GPT2, PSAT1, ASNS, OAT, PSPH, ALDH18A1, and CCBL1.

In operation 1120, the determination unit 120 determines a possibility of cancer of the subject 3 by using the acquired first gene expression data and the second gene expression data of the normal person group 1 and the cancer patient group 2. Here, the second gene expression data may be pre-stored in the storage unit 130 along with the discriminant model which is pre-generated based on the second gene expression data. The determination unit 120 reads out from the second gene expression data or the discriminant model pre-stored in the storage unit 130, and determines a possibility of cancer by using the readout second gene expression data or discriminant model.

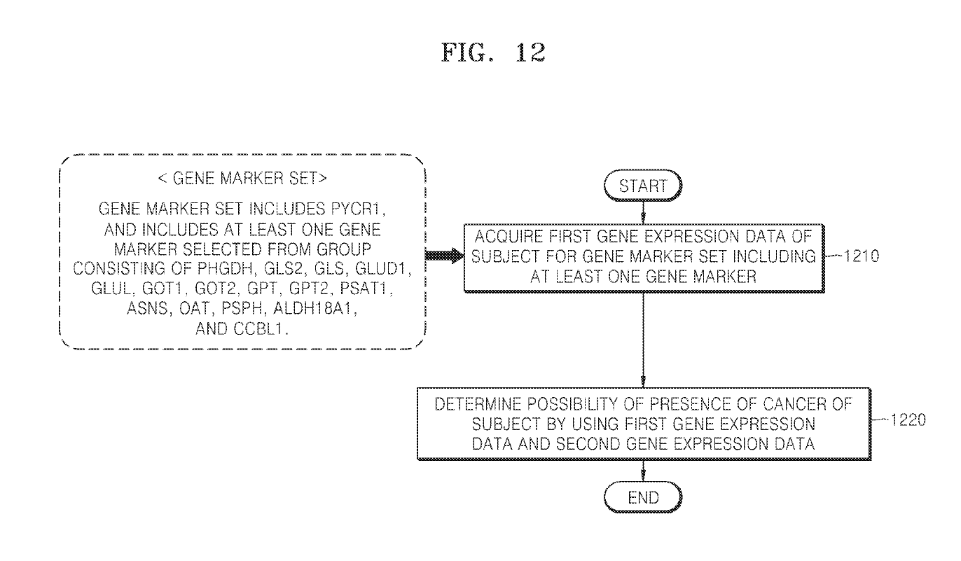

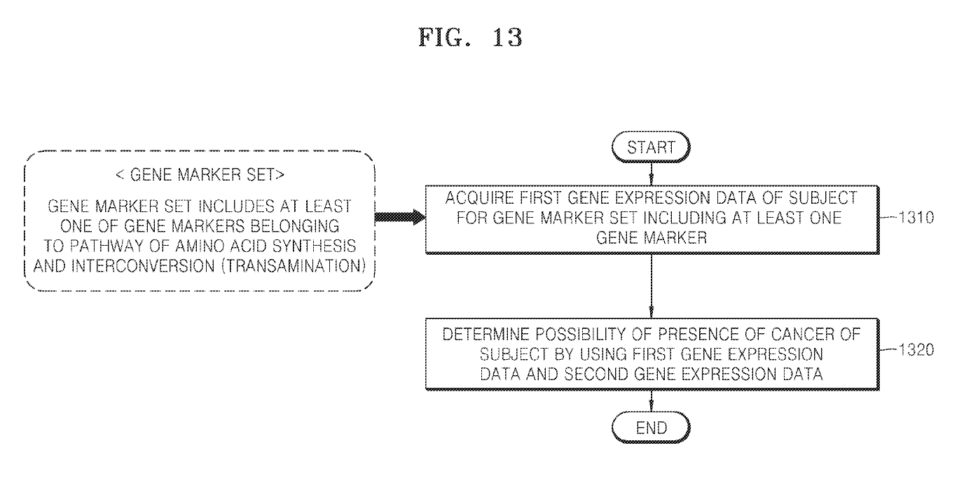

FIG. 12 is a flowchart of a method of diagnosing cancer by using genetic information, according to another embodiment of the present invention. Referring to FIG. 12, the cancer diagnosis method according to the present embodiment includes a plurality of operations which are performed in a time series in the cancer diagnostic system 100 and the cancer diagnostic apparatus 10 illustrated in FIGS. 1, 7, and 8. Therefore, although not described below, descriptions above with reference to the drawings may be applied to the cancer diagnosis method according to the present embodiment.

In operation 1210, the gene expression data acquiring unit 110 acquires the first gene expression data of the subject 3 for a gene marker set including at least one gene marker.

The gene marker set used in operation 1210 includes PYCR1, and includes at least one gene marker selected from the group consisting of PHGDH, GLS2, GLS, GLUD1, GLUL, GOT1, GOT2, GPT, GPT2, PSAT1, ASNS, OAT, PSPH, ALDH18A1, and CCBL1.

In operation 1220, the determination unit 120 determines a possibility of cancer of the subject 3 by using the acquired first gene expression data and the second gene expression data of the normal person group 1 and the cancer patient group 2. Here, the second gene expression data may be pre-stored in the storage unit 130 along with the discriminant model which is pre-generated based on the second gene expression data. The determination unit 120 reads out from the second gene expression data or the discriminant model pre-stored in the storage unit 130, and determines a possibility of cancer by using the readout second gene expression data or discriminant model.

FIG. 13 is a flowchart of a method of diagnosing cancer by using genetic information, according to another embodiment of the present disclosure. Referring to FIG. 13, the cancer diagnosis method according to the present embodiment includes a plurality of operations which are performed in a time series in the cancer diagnostic system 100 and the cancer diagnostic apparatus 10 illustrated in FIGS. 1, 7, and 8. Therefore, although not described below, descriptions above with reference to the drawings may be applied to the cancer diagnosis method according to the present embodiment.

In operation 1310, the gene expression data acquiring unit 110 acquires the first gene expression data of the subject 3 for a gene marker set including at least one gene marker.

The gene marker set used in operation 1310 includes at least one of the gene markers belonging to the pathway of amino acid synthesis and interconversion (transamination).

In operation 1320, the determination unit 120 determines a possibility of cancer of the subject 3 by using the acquired first gene expression data and the second gene expression data of the normal person group 1 and the cancer patient group 2. Here, the second gene expression data may be pre-stored in the storage unit 130 along with the discriminant model which is pre-generated based on the second gene expression data. The determination unit 120 reads out from the second gene expression data or the discriminant model pre-stored in the storage unit 130, and determines a possibility of cancer by using the readout second gene expression data or discriminant model.

As described above, according to the one or more of the above embodiments of the present invention, cancer is diagnosed by using only genetic information of a person. In particular, cancer is accurately diagnosed by using gene markers highly associated with the occurrence of a disease.

The above-described embodiments of the present invention may be written as computer programs and may be implemented in general-use digital computers that execute the programs using a computer-readable recording medium. Also, a structure of data used in the aforementioned embodiments may be recorded in computer-readable recording media through various members. Examples of the computer-readable recording medium include magnetic storage media (e.g., ROM, floppy disks, hard disks, etc.), optical recording media (e.g., CD-ROMs or DVDs), etc), and transmission media such as Internet transmission media.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of diagnosing cancer by using genetic information, the method comprising:
acquiring first gene expression data of a subject, for whom cancer is to be diagnosed, for a gene marker set including at least one gene marker; and
determining a possibility of a presence of the cancer of the subject by using the acquired first gene expression data and pre-stored second gene expression data of a normal person group and a cancer patient group,
wherein the gene marker set comprises at least one gene marker selected from the group consisting of pyrroline-5-carboxylate reductase 1 (PYCR1), phosphoglycerate dehydrogenase (PHGDH), glutaminase 2 (liver, mitochondrial) (GLS2), glutaminase (GLS), glutamate dehydrogenase 1 (GLUD1), glutamate-ammonia ligase (GLUL), glutamic-oxaloacetic transaminase 1 and soluble (aspartate aminotransferase 1) (GOT1), glutamic-oxaloacetic transaminase 2 and mitochondrial (aspartate aminotransferase 2) (GOT2), glutamic-pyruvate transaminase (alanine aminotransferase) (GPT), glutamic pyruvate transaminase (alanine aminotransferase 2) (GPT2), phosphoserine aminotransferase 1 (PSAT1), asparagine synthetase (glutamine-hydrolyzing) (ASNS), ornithine aminotransferase (OAT), phosphoserine phosphatase (PSPH), aldehyde dehydrogenase 18 family and member A1 (ALDH18A1), and cysteine conjugate-beta lyase cytoplasmic (CCBL1).

2. The method of claim 1, wherein the gene marker set comprises PYCR1, and further comprises at least one gene marker selected from the group consisting of PHGDH, GLS2, GLS, GLUD1, GLUL, GOT1, GOT2, GPT, GPT2, PSAT1, ASNS, OAT, PSPH, ALDH18A1, and CCBL1.

3. The method of claim 1, wherein the gene marker set comprises one gene marker PYCR1.

4. The method of claim 1, wherein the gene marker set comprises all the gene markers PYCR1, PHGDH, GLS2, GLS, GLUD1, GLUL, GOT1, GOT2, GPT, GPT2, PSAT1, ASNS, OAT, PSPH, ALDH18A1, and CCBL1.

5. The method of claim 1, further comprising preprocessing the first gene expression data including first gene expression levels, based on a distribution of second gene expression levels included in the pre-stored second gene expression data,
wherein the determining the possibility of the presence of the cancer comprises determining presence of the cancer by using the preprocessed first gene expression data and the pre-stored second gene expression data.

6. The method of claim 5, wherein the preprocessing comprises calculating ratios of the second gene expression levels and the first gene expression levels in units of a gene marker to preprocess the first gene expression data.

7. The method of claim 5, wherein the preprocessing comprises normalizing or standardizing the first gene expression levels in units of a gene marker to preprocess the first gene expression data in comparison with the second gene expression levels.

8. The method of claim 5, wherein the determining comprises applying the preprocessed first gene expression data to a discriminant model, pre-generated from the pre-stored second gene expression data, to determine the possibility of cancer.

9. The method of claim 8, wherein the discriminant model is pre-generated by using a regression model, which has a univariate representing the gene marker set or a multi-variate corresponding to two or more of the gene markers included in the gene marker set, for the pre-stored second gene expression data.

10. The method of claim 8, wherein,
the determining the possibility of the presence of the cancer comprises:
calculating an index indicating a degree of expression of the first gene expression levels in the preprocessed first gene expression data for the second gene expression levels; and
applying the calculated index to the pre-generated discriminant model to calculate a statistical significance level indicating the possibility of the presence of the cancer.

11. The method of claim 10, wherein the calculating of an index comprises calculating the index by using at least one of the following methods: a fisher exact test, a binomial test, a geneset enrichment analysis (GSEA), a Mahalanobis distance, an Euclid distance, a Manhattan distance, a maximum distance, a minimum distance, and a correlation coefficient.

12. The method of claim 10, wherein,
the calculating the index comprises estimating a representative expression pattern that is obtained by summarizing distributions of third gene expression levels of the normal person group in the second gene expression data, and
the index is calculated based on the degree of expression of the first gene expression levels in the preprocessed first gene expression data for the estimated representative expression pattern.

13. The method of claim 5, wherein,
the determining the possibility of the presence of the cancer comprises:
calculating an index, indicating a degree of expression of the first gene expression levels in the preprocessed first gene expression data, for a representative expression pattern that is obtained by summarizing distributions of third gene expression levels of the normal person group; and
calculating a statistical significance level indicated by the calculated index by using an empirical distribution of degrees of expression of the third gene expression levels for the representative expression pattern.

14. The method of claim 10, wherein,
the determining the possibility of the presence of the cancer further comprises comparing the calculated statistical significance level and a threshold value, which is used to determine the presence of cancer or a degree of occurrence of cancer, by using the pre-generated discriminant model, and
the possibility of cancer is determined based on the comparison result.

15. An apparatus for diagnosing cancer by using genetic information, the apparatus comprising:
a gene expression data acquiring unit that acquires first gene expression data of an subject, for whom cancer is to be diagnosed, for a gene marker set including at least one gene marker; and
a determination unit that determines a possibility of cancer of the subject by using the acquired first gene expression data and pre-stored second gene expression data of a normal person group and a cancer patient group,
wherein the gene marker set comprises at least one gene marker selected from the group consisting of pyrroline-5-carboxylate reductase 1 (PYCR1), phosphoglycerate dehydrogenase (PHGDH), glutaminase 2 (liver, mitochondrial) (GLS2), glutaminase (GLS), glutamate dehydrogenase 1 (GLUD1), glutamate-ammonia ligase (GLUL), glutamic-oxaloacetic transaminase 1 and soluble (aspartate aminotransferase 1) (GOT1), glutamic-oxaloacetic transaminase 2 and mitochondrial (aspartate aminotransferase 2) (GOT2), glutamic-pyruvate transaminase (alanine aminotransferase) (GPT), glutamic pyruvate transaminase (alanine aminotransferase 2) (GPT2), phosphoserine aminotransferase 1 (PSAT1), asparagine synthetase (glutamine-hydrolyzing) (ASNS), ornithine aminotransferase (OAT), phosphoserine phosphatase (PSPH), aldehyde dehydrogenase 18 family and member A1 (ALDH18A1), and cysteine conjugate-beta lyase cytoplasmic (CCBL1),
wherein the gene expression data acquiring unit and the determination unit are implemented by at least one processor.

16. The apparatus of claim 15, wherein the gene marker set comprises PYCR1, and comprises at least one gene marker selected from the group consisting of PHGDH, GLS2, GLS, GLUD1, GLUL, GOT1, GOT2, GPT, GPT2, PSAT1, ASNS, OAT, PSPH, ALDH18A1, and CCBL1.

17. The apparatus of claim 15, wherein,
the determination unit comprises a preprocessor that preprocesses the first gene expression data including first gene expression levels, based on a distribution of second gene expression levels included in the pre-stored second gene expression data, and
the determination unit determines the presence of cancer by using the preprocessed first gene expression data and the pre-stored second gene expression data.

18. The apparatus of claim 17, further comprising a storage unit that stores a discriminant model pre-generated from the pre-stored second gene expression data,
wherein the determination unit determines the possibility of cancer by using the pre-generated discriminant model and the preprocessed first gene expression data.

19. The apparatus of claim 18, wherein,
the determination unit further comprises a calculator that calculates an index, indicating a degree of expression of the first gene expression levels in the preprocessed first gene expression data, for the second gene expression levels, and applies the calculated index to the pre-generated discriminant model to calculate a statistical significance level indicating a presence probability of the cancer, and
the determination unit determines the possibility of cancer, based on the calculated statistical significance level.

20. The apparatus of claim 19, wherein,
the determination unit further comprises a comparator that compares the calculated statistical significance level and a threshold value, which is used to determine the presence of cancer or a degree of occurrence of cancer, by using the pre-generated discriminant model, and
the determination unit determines the possibility of cancer, based on the comparison result.

21. A method of detecting cancer using genetic information, the method comprising:
acquiring first gene expression data of a subject for a gene marker set including at least one gene marker; and
comparing the acquired first gene expression data to pre-stored second gene expression data of a normal person group and a cancer patient group by calculating statistical similarity of the first gene expression data to the pre-stored second gene expression data, wherein cancer in the subject is indicated if the first gene expression data is more similar to the pre-stored second gene expression data of the cancer patient group than to the pre-stored second gene expression data of the normal patient group, and
wherein the gene marker set comprises at least one gene marker selected from the group consisting of pyrroline-5-carboxylate reductase 1 (PYCR1), phosphoglycerate dehydrogenase (PHGDH), glutaminase 2 (liver, mitochondrial) (GLS2), glutaminase (GLS), glutamate dehydrogenase 1 (GLUD1), glutamate-ammonia ligase (GLUL), glutamic-oxaloacetic transaminase 1 and soluble (aspartate aminotransferase 1) (GOT1), glutamic-oxaloacetic transaminase 2 and mitochondrial (aspartate aminotransferase 2) (GOT2), glutamic-pyruvate transaminase (alanine aminotransferase) (GPT), glutamic pyruvate transaminase (alanine aminotransferase 2) (GPT2), phosphoserine aminotransferase 1 (PSAT1), asparagine synthetase (glutamine-hydrolyzing) (ASNS), ornithine aminotransferase (OAT), phosphoserine phosphatase (PSPH), aldehyde dehydrogenase 18 family and member A1 (ALDH18A1), and cysteine conjugate-beta lyase cytoplasmic (CCBL1),
wherein the statistical similarity is calculated using at least one of a fisher exact test, a binomial test, a geneset enrichment analysis (GSEA), a Mahalanobis distance, a Euclid distance, a Manhattan distance, a maximum distance, a minimum distance, and a correlation coefficient.
